(19) United States
(12) Patent Application Publication
(10) Pub. No.: US 2017/0049477 A1
(43) Pub. Date: Feb. 23, 2017

(54) Apparatus Adapted for Resiliently Holding the Resected Fibular Sections for Preventing Rejoining Thereof

(71) Applicant: Paonan Biotech co., Ltd., Taipei City (TW)

(72) Inventor: Chung-Chun Yeh, Taipei City (TW)

(73) Assignee: Paonan Biotech co., Ltd.

(21) Appl. No.: 15/216,421

(22) Filed: Jul. 21, 2016

(30) Foreign Application Priority Data
Aug. 18, 2015 (TW) 104126952

(51) Int. Cl. A61B 17/68; A61F 2/28

(52) U.S. Cl. CPC A61B 17/68; A61F 2/2814; A61F 2002/285; A61B 2017/00694

(57) ABSTRACT

An apparatus, adapted for resiliently holding resected fibula sections, comprises: a first connecting portion adapted for connecting a first resected fibular section of a patient's fibula; a second connecting portion adapted for connecting a second resected fibular section of the patient's fibula; and an elastic portion or member defined between the first connecting portion and the second connecting portion adapted for resiliently holding the first resected fibular section and the second resected fibular section, whereby upon actuation by an impacting force caused by the patient's movement, the elastic portion may resiliently buffer such an impacting force to prevent contacting or rejoining of the first and second resected fibular sections.

### RELATED APPLICATION

This application claims the benefit of a Taiwanese patent application, 104126952, filed on Aug. 18, 2015, the specification of which is incorporated here by this reference.

### BACKGROUND OF THE INVENTION

Prof. Zhang, Ying-Ze, Department of Orthopedic Surgery, the Third Hospital of Hebei Medical University, China, ever disclosed a theory of “Non-uniform Settlement of Knee Joint in the Treatment of Osteoarthristis”. In his theory, “non-uniform settlement” of tibial plateau plays a key role in the development of knee joint osteoarthritis (OA). Reviewing his theory, a fibula is a tubular cortical bone with high bone density, when compared with the proximal tibia, which consists most of cancellous bone with a large weight-bearing area without bony barrier in the medial side. However, when the medial side of the tibia is attacked with osteoporosis, the rigid fibular support in the lateral side to (or a supporting force by the fibula acting upon) the osteoporotic proximal tibia may contribute to a non-uniform settlement of tibial plateau, thereby shifting a mechanical axis, aggravating weight-bearing in the medial plateau, resulting in articular cartilage degeneration and knee varus to cause knee pain. By the way, a partial fibular osteotomy was done by resecting a 2 cm-long section of fibula at a location 6˜10 cm below the fibula head to treat medial compartment knee osteoarthritis (OA) to reduce the knee pain significantly in the varus osteoarthritic knee. The resected fibula may not stress the proximal tibia to thereby relieve or reduce the knee joint pain.

Even though the resected fibula may temporarily reduce the knee pain of medial compartment knee OA, such resected sections of fibula may still be contacted, especially when subjected to compression or pressure due to a patient's knee movement, to be rejoined due to bone regrowth or fusion of the resected fibular sections. The rejoined fibula will restore its supporting force to bias the proximal tibia to cause again the “non-uniform settlement” of the tibial plateau, thereby still causing the patient's knee pain.

The present inventor has found the drawbacks of the prior technique, and invented the present apparatus for preventing rejoining of the resected fibular sections.

### SUMMARY OF THE INVENTION

The object of the present invention is to provide an apparatus, adapted for resiliently holding resected fibula sections, comprising: a first connecting portion adapted for connecting a first resected fibular section of a patient's fibula; a second connecting portion adapted for connecting a second resected fibular section of the patient's fibula; and an elastic portion or member defined between the first connecting portion and the second connecting portion adapted for resiliently holding the first resected fibular section and the second resected fibular section, whereby upon actuation by an impacting force caused by the patient's movement, the elastic portion may resiliently buffer such an impacting force to prevent contacting or rejoining of the first and second resected fibular sections, and to prevent from the non-uniform settlement of the tibial plateau caused by a supporting force due to the rejoined fibular sections.

### BRIEF DESCRIPTION OF THE DRAWINGS

FIG. 1 is a perspection drawing showing a first preferred embodiment of the present invention.

FIG. 2 is an illustration showing the application of the present invention as shown in FIG. 1.

FIG. 3 shows a second preferred embodiment of the present invention.

FIG. 4 shows an application of the embodiment of FIG. 3.

FIG. 5 shows a third preferred embodiment of the present invention.

FIG. 6 shows a fourth preferred embodiment of the present invention.

FIG. 7 shows a fifth preferred embodiment of the present invention.

FIG. 8 shows a sixth preferred embodiment of the present invention.

FIG. 9 shows a seventh preferred embodiment of the present invention.

FIG. 10 shows an eighth preferred embodiment of the present invention.

FIG. 11 is a sectional illustration of the embodiment of FIG. 10.

FIG. 12 shows a ninth preferred embodiment of the present invention.

FIG. 13 is an illustration showing the application of FIG. 12.

FIG. 14 shows a tenth embodiment of the present invention.

FIG. 15 is an illustration showing the application of FIG. 14 as compressed.

FIG. 16 is an illustration of the application when resiliently expanded from FIG. 15.

FIG. 17 shows an eleventh embodiment of the present invention.

FIG. 18 shows a twelveth embodiment of the present invention.

FIG. 19 is an illustration as derived from FIG. 18 by downwardly extending the second connecting portion of the present invention.

### DETAILED DESCRIPTION

As shown in the drawing figures of the present invention, an apparatus adapted for resiliently holding the resected fibular sections for preventing rejoining thereof comprises: a first connecting portion 11 adapted for connecting a first resected fibular section F1 (proximal to the fibula head) of a patient's fibula F; a second connecting portion 12 adapted for connecting a second resected fibular section F2 of the patient's fibula; and an elastic portion (or member) 13 defined between the first connecting portion 11 and the second connecting portion 12 adapted for resiliently holding the first resected fibular section F1 and the second resected fibular section F2.

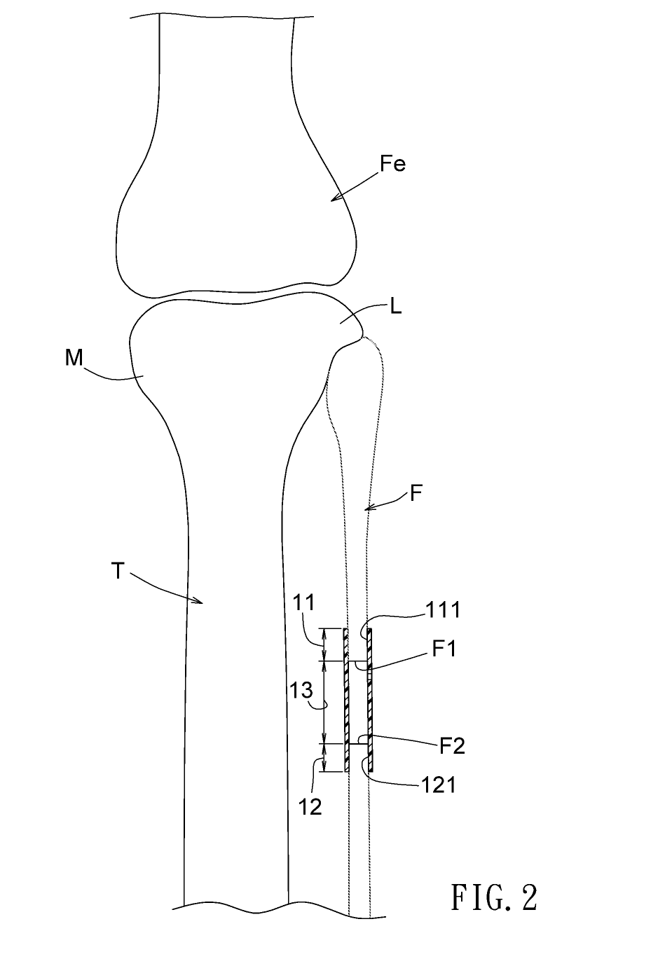

When subjected to a compression force such as due to the gravitational force of the patient, the compression depressing the resected fibular section or sections will be resiliently buffered by the elastic portion 13 between the first connecting portion 11 as coupled with the first resected fibular section F1 and the second connecting portion 12 as coupled with the second resected fibular section F2 of the patient. In other words, the resected fibular sections F1, F2 are no longer connected and are resiliently held by the elastic portion 13 of the present invention, so that the “fibula” (as being resected) F, being not an original rigid fibula bone, will not rigidly support the lateral portion L of the tibia T (or the tibial head) as shown in FIG. 2 to cause the “non-uniform settlement” as found in a medial compartment knee osteoarthritis (OA). The patient's weight as downwardly loading from the femur Fe towards the tibia T will be uniformly distributed on the medial portion M and the lateral portion L of the tibia T (the biasing force or the supporting force from the fibular side already removed due to the resected fibular sections) to thereby release the stress on the medial portion (or side) M so as to releave or reduce the medial compartment knee OA pain.

Otherwise, if the resected fibular sections F1, F2 were accidentally contacted and rejoined due to bone regrowth or fusion, the rejoined fibula F will urge the lateral portion (or side) L of the tibial head to “bias” the medial portion (or medial side) M to cause the so-called “non-uniform settlement” of the tibial plateau, thereby resulting in medial compartment knee OA or knee pain.

Fortunately, after the resilient holding of the resected fibular sections F1, F2 by the elastic portion 13 of the present invention, the two resected fibular sections F1, F2 are disconnected, but resiliently held or limited in the apparatus of the present invention, like being captured in a “cage”. So, the resected fibular sections F1, F2 will not be contacted and rejoined to thereby remove the nidus of the medial compartment OA pain and releave the knee pain. The resected fibular sections F1, F2 will be stably held within such a “cage” and will no longer swing or vibrate freely so as to comfort the patient since he or she may not worry about any random moving of the resected fibular sections.

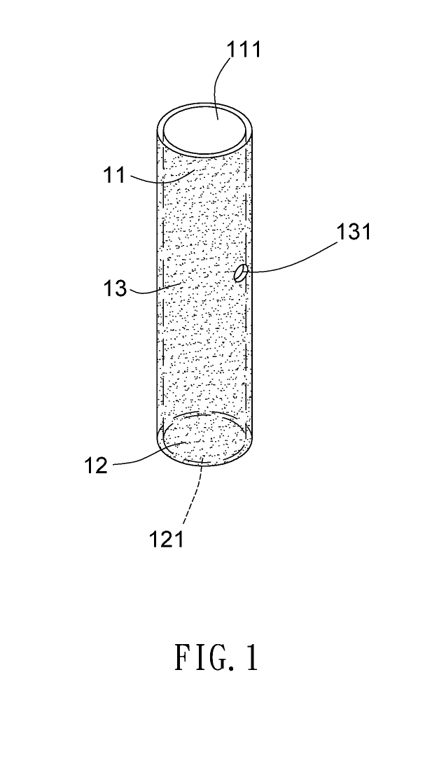

As shown in FIGS. 1 and 2, the present invention is formed as a tubular member made of elastomeric materials, including a first connecting portion 11 having a first cavity 111 recessed in the first connecting portion 11 adapted for sheathing or fastening the first resected fibular section F1 of the fibula F in the first cavity 111; a second connecting portion 12, opposite to the first connecting portion 11, having a second cavity 121 adapted for sheathing or fastening the second resected fibular section F2 of the fibula F; and an elastic portion 13 defined between the first and the second connecting portions 11, 12.

The first resected fibular section F1 is fastened in the upper or first cavity 111, while the second resected fibular section F2 is fastened on the lower or second cavity 121, so that both fibular sections F1, F2 are separated without being contacted or touched. When subjected to any compression or external force caused by the patient's movements, the elastic portion 13 between the first connecting portion 11 and the second connecting portion 12 will be resiliently deformed or flexibly bent to buffer the external force or compression impacting upon the fibular sections, thereby preventing from contact or rejoining of the two resected fibular sections F1, F2 due to bone regrowth or fusion.

As shown in FIGS. 1, 2 and 3, the tubular member of the present invention is integrally formed with elastomers including silicon rubber to be a hollow tube (or hose) having a first cavity 111 axially formed in an upper portion of the first connecting portion 11, a second cavity 121 axially formed in a lower portion of the second connecting portion 12, and the elastic portion 13 defined between the first and second connecting portions 11, 12.

As shown in FIG. 1, a through hole 131 is laterally or radially formed through the tubular member of the present invention for releasing tissue fluid from inside the tubular member, or for filling or injecting inhibitor into the tubular member for inhibiting bone regrowth or fusion such as hydrogel or silicongel. The through hole 131 may also be eliminated to form a closed tubular member.

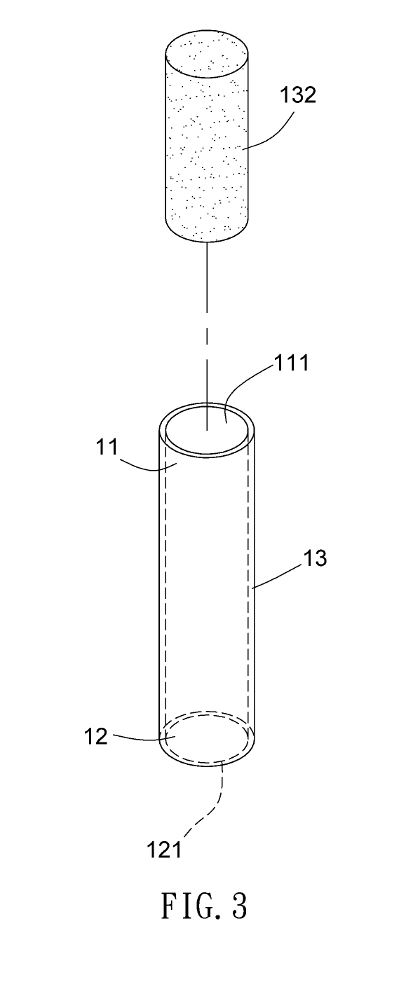

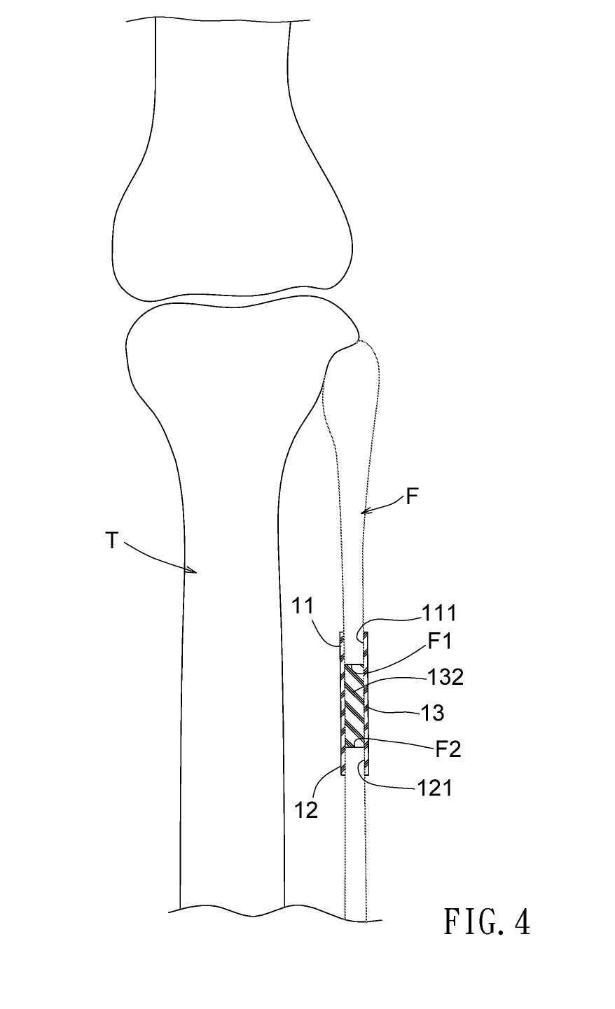

As shown in FIGS. 3 and 4, an elastomeric column 132 is inserted into the hollow tubular member of the present invention adapted to be resiliently retained in between the first and the second resected fibular sections F1, F2 to prevent from contacting, bone regrowth or rejoining of the two resected fibular sections. The elastomeric column 132 may be made of different coefficients of elasticity.

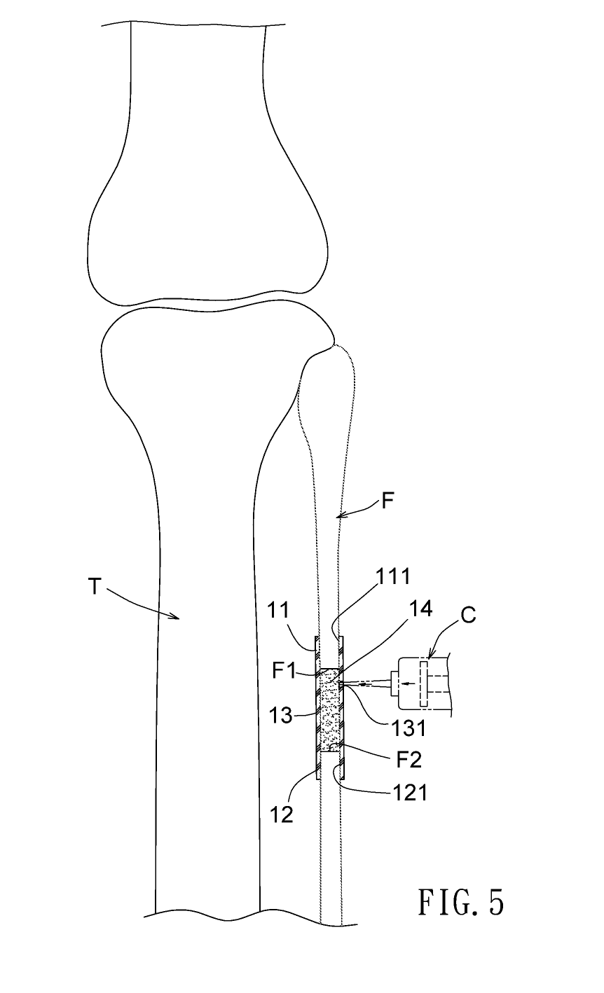

In FIG. 5, an injector or syringe C is provided to inject a filling material 14 or an inhibitor for inhibiting bone regrowth or fusion of the resected fibular sections F1, F2 through the through hole 131 formed in the elasted portion 13 of the present invention. Such a filling material may include silicon gel, silicon rubber or hydrogel, etc.

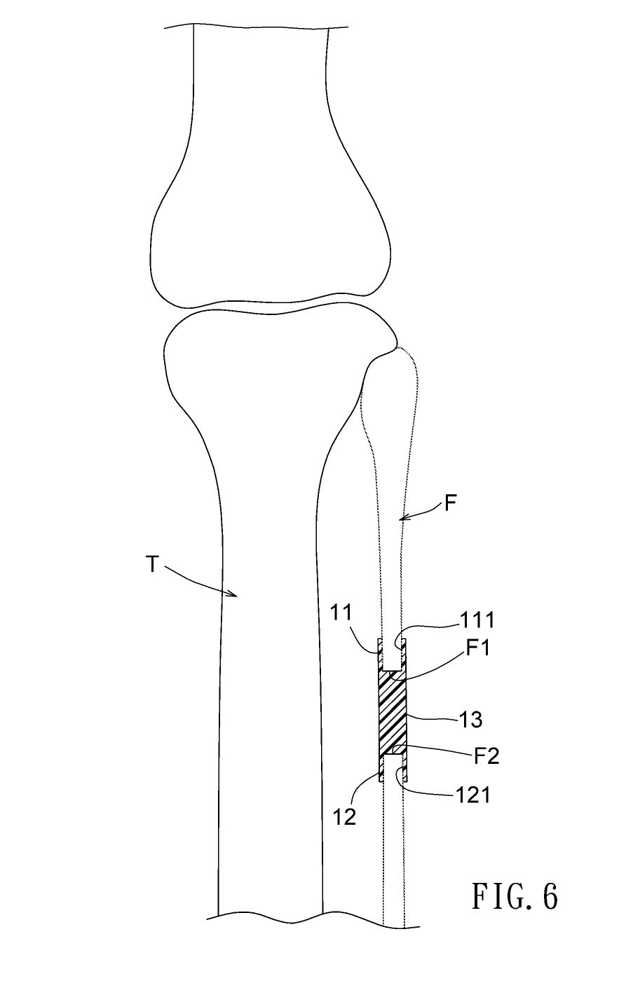

In FIG. 6, the first connecting portion 11, the elastic portion 13, and the second connecting portion 12 are integrally formed, such as made of silicon rubber or other elastomers. A first cavity 111 is axially recessed inwardly in the first or upper end of the first connecting portion 11 adapted to sheathe the first resected fibular section F1 into the first cavity 111, and a second cavity 121 is axially recessed inwardly in the second or lower end of the second connecting portion 12 adapted to sheathe the second resected fibular section F2 into the second cavity 121. Such an integrally formed elastomeric rod member is “solid”, not a hollow core, and thus really “disconnect” the two resected fibular sections F1, F2, thereby preventing from their rejoining.

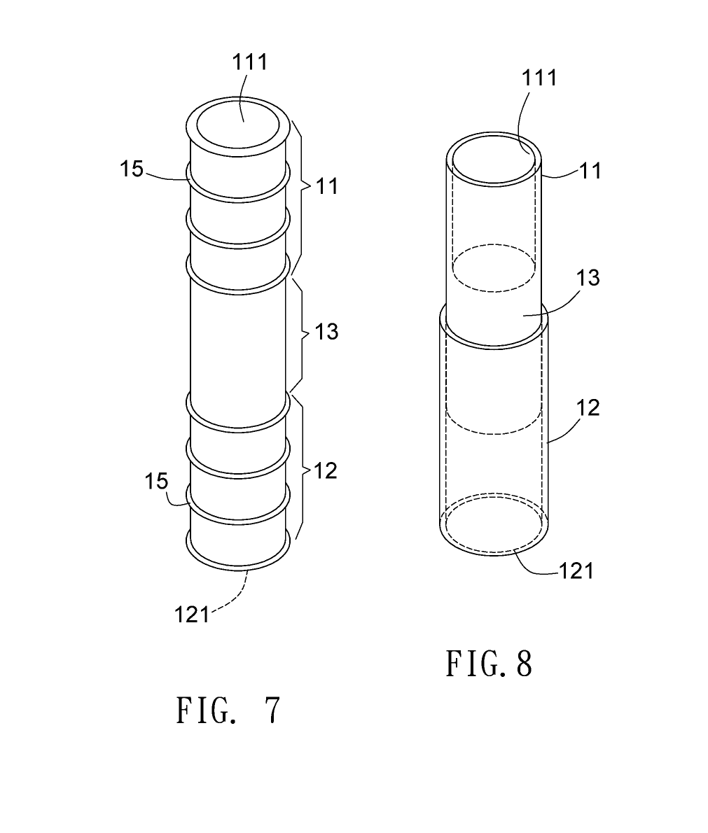

Similarly, an integrally formed elastomeric rod member of the present invention as shown in FIG. 7, as modified from that of FIG. 6, further includes a plurality of extension rings 15 respectively circumferentially concentrically formed on the first connecting portion 11, and the second connecting portion 12. Such extension rings 15 serve as reinforcing rings for protecting the rod member of the present invention and also serve as “scales” corresponding to the length or height of the rod member so that the rod member can be cut off along a specific extension ring 15 to meet the specific size as required by a patient.

In FIG. 8, a first connecting portion 11 (having first cavity 111 formed therein) is integrally formed with an elastic portion 13 to from as a “plunger” which is slidably held in a hollow second connecting portion 12 (having a second cavity 121 formed in the second connecting portion 12) so as to be slidably adjusted for the length or height of the present invention to meet the patient's requirements, such as for his or her preferred length or height.

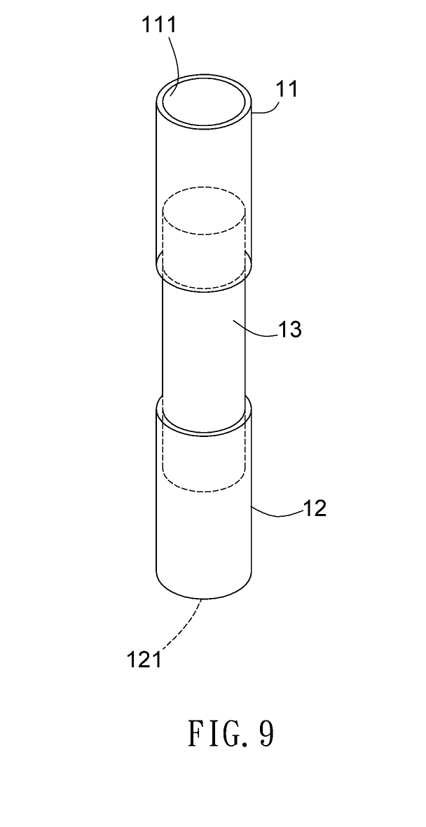

In FIG. 9, the elastic portion 13 is an elastomeric cylindrical (or rod) member, slidably adjustably sheathed or held in a hollow first connecting portion 11, and a hollow second connecting portion 12 to adjustably meet for the patient's requirement.

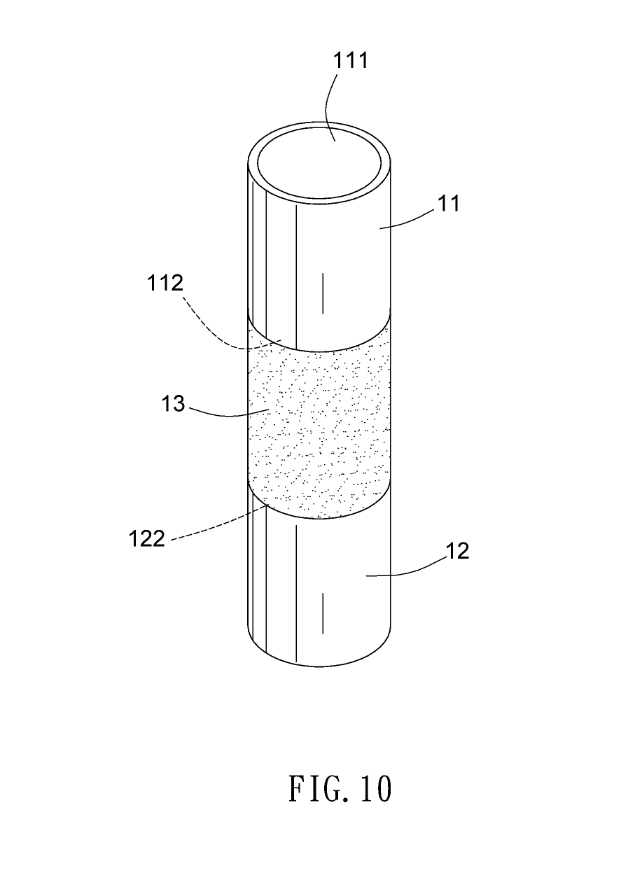

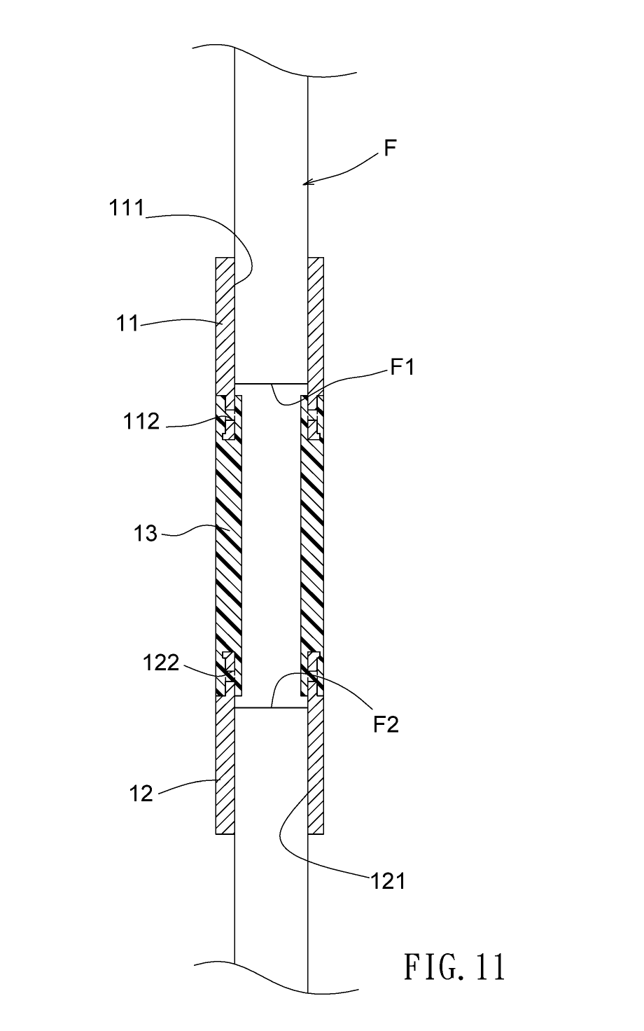

As shown in FIGS. 10 and 11, the present invention is modified to include an elastic portion 13 formed as an elastomeric cylindrical (or rod) member and embeddedly connected with the first connecting portion 11 and with the second connecting portion 12. Each connecting portion 11 or 12 is made of metals (including titanium alloy), polymers, ceramic or composite materials. The elastic portion 13 may be made of silicon rubber or other elastomeric materials. Each connecting portion 11 or 12 is formed with an embedding part 112 or 122 in order to be embedded with a fastening groove formed in an upper or lower periphery of the elastic portion 13 for firmly embedding the elastic portion 13 with the first and second connecting portions 11, 12.

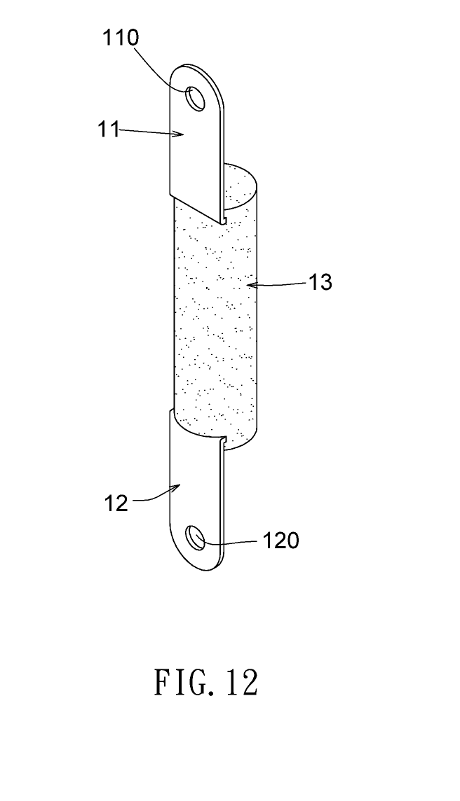

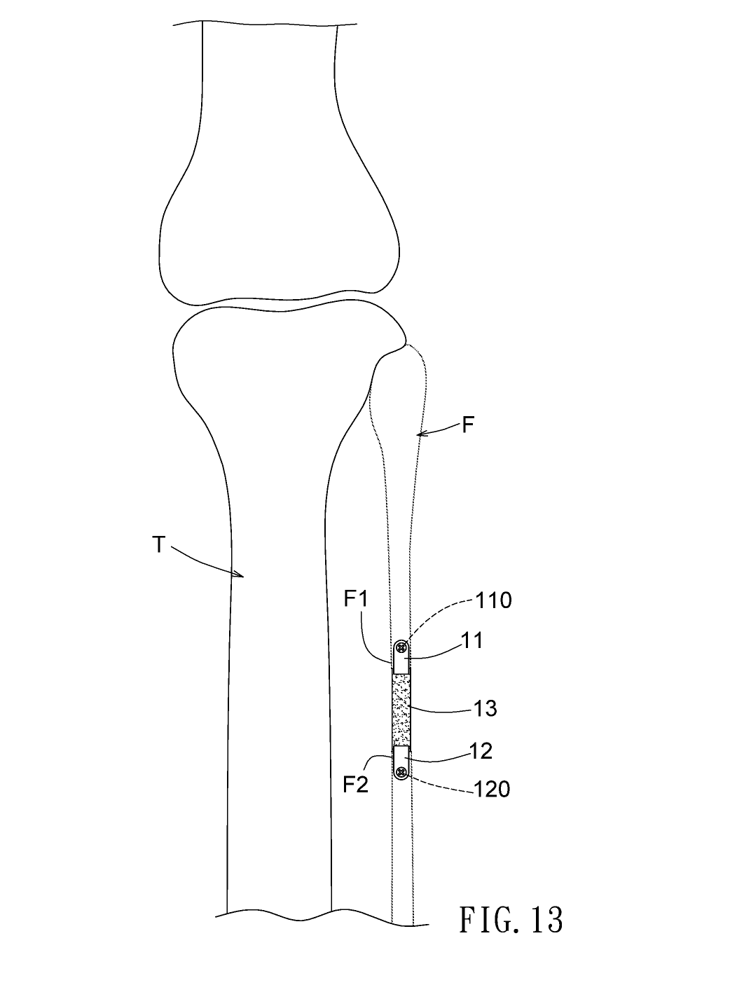

As shown in FIGS. 12 and 13, the present invention comprises an elastic portion 13 which may be formed as an elastic member or elastic cylindrical member, a first connecting portion 11 secured to a first end portion or upper end portion of the elastic portion 13 and adapted to be fastened to a first resected fibular section F1 by fixing a screw through a fixing hole 110 formed through the first connecting portion 11, and a second connecting portion 12 secured to a second or lower end portion of the elastic portion 13 and adapted to be fastened to a second resected fibular section F2 by fixing a screw through a fixing hole 120 formed through the second connecting portion 12. Each connecting portion 11, 12 may be formed as a bracket as shown in FIG. 12.

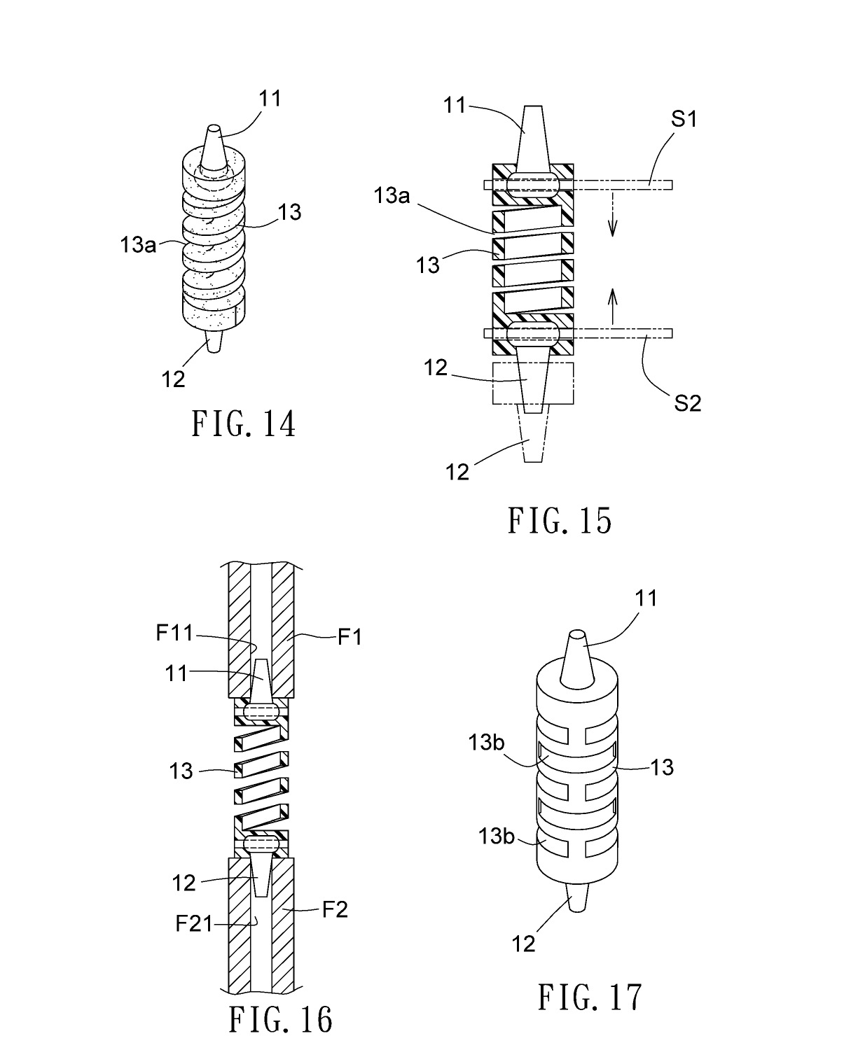

As shown in FIGS. 14-16, the present invention may be modified to include a first connecting portion 11 which is formed as a first peg (or projection) tapered or protruded upwardly adapted to be fastened into a first medullary cavity F11 in the first resected fibular section F1, and a second connecting portion 12 which is formed as a second peg (or projection) tapered or protruded downwardly adapted to be fastened into a second medullary cavity F21 in the second resected fibular section F2.

In FIGS. 14-16, the elastic portion 13 is modified to be a helical-spring structure composed of a plurality of helical spring coils 13a helically wound, and retained between the first connecting portion 11 (formed as a peg), and the second connecting portion 12 (also as a peg). A pair of tools S1, S2 is respectively inserted into a pair of holes formed in the first and second connecting portion 11, 12. Then, compressing the two tools S1, S2, to compress the spring coils 13a to retract the two pegs 11, 12 to be positioned in between the first and second fibular section F1, F2. After releasing the two tools S1, S2, the spring coils 13a will be expandably restored to insert the two pegs 11, 12 into the two medullary cavities F11, F12 of the two fibular sections F1, F2 to stably fasten the present invention between the first and second resected fibular sections F1, F2 of the fibula F (FIG. 16).

In FIG. 17, the elastic portion 13 is modified to be a cylindrical elastic member having a plurality of compressible slots or grooves 13b circumferentially recessed in the cylindrical elastic member 13. Such slots or grooves 13b allow the elastic member to be compressible and expandable so that the present invention may be first compressibly positioned in between the two fibular sections F1, F2 and then expandably fastened into the medullary cavities of the two fibular sections F1, F2 for firmly fastening the present invention in the resected fibular sections.

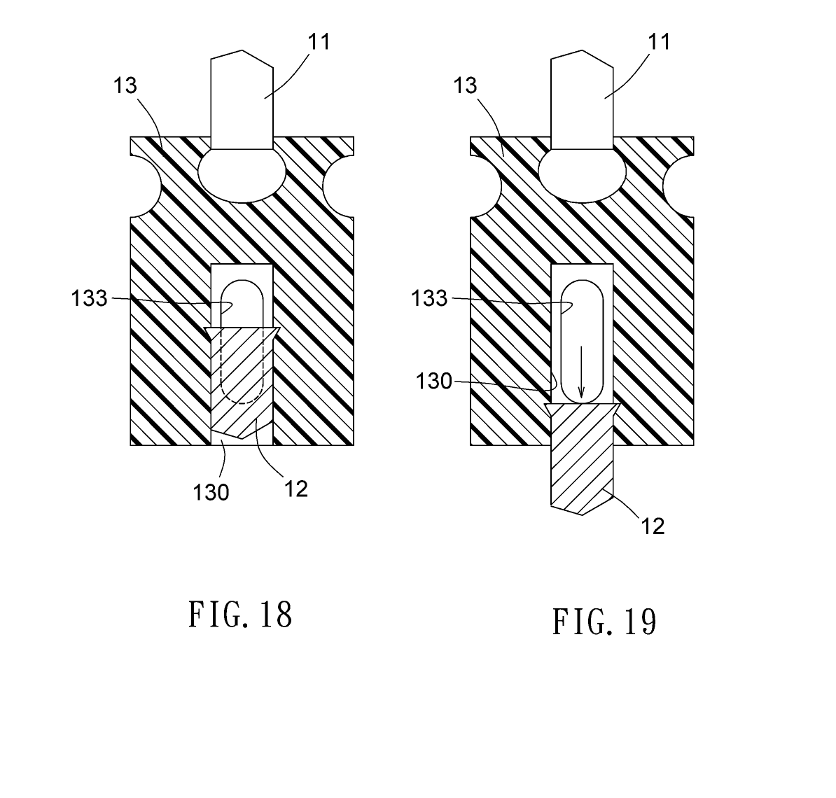

As shown in FIGS. 18 and 19, the first connecting portion 11 is embedded in an upper portion of the elastic portion 13, adapted to be fastened to the first fibular section F1; and the second connecting portion 12 is slibalby adjustably formed in a lower portion of the elastic portion 13. The elastic portion 13 has a central cylindrical hole 130 axially formed in a lower portion of the elastic portion 13 to allow the second connecting portion 12 which is formed as a plunger to be slidably adjustably reciprocated in the central cylindrical hole 130; and a longitudinal slot 133 diametrically formed through the elastic portion 13 and communicated with the cylindrical hole 130 adapted to insert a tool (not shown) through the slot 133 for pushing the second connecting portion 12 (now formed as a plunger) downwardly to be fastened into the medullary cavity F21 of the second resected fibular section F2.

The present invention has the following advantages:
1. The resected fibular sections F1, F2 have been resiliently held in the elastic portion 13 of the present invention to prevent from their rejoining or bone-regrowth, to thereby eliminate a supporting force of a rejoined fibula which may cause non-uniform settlement of the tibia. Therefore, the medial compartment knee osteoarthritis or knee pain of a patient may thus be prevented.
2. The resected fibular ends have been held within the elastic portion of the present invention so that the resected fibular sections F1, F2 will not be freely moved, vibrated, swung or oriented to injure or wound the nearby tissue.
3. The patient may be comforted psychologically since his or her resected fibular sections had been stably held in the elastic portion of the present invention. So, he or she will not worry about the free movement or unexpected result caused by such resected fibular sections.

The present invention may be further modified without departing from the spirit and scope of the present invention

I claim:

1. An apparatus, adapted for resiliently holding a patient's resected fibular sections, comprising:
an elastic member made of elastomeric materials;
a first connecting portion formed on a first end portion of the elastic member and adapted for fastening or connecting a first resected fibular section, proximal to the patient's fibula head, of the patient's fibula; and
a second connecting portion formed on a second end portion of the elastic member, opposite to the first connecting portion, and adapted for fastening or connecting a second resected fibular section of the patient's fibula;
whereby said elastic member will buffer an external force or vibration caused by the patient's movement and will prevent the resected fibular sections from being rejoined due to unexpected bone regrowth or fusion.

2. An apparatus according to claim 1, wherein each said connecting portion is respectively embedded into said elastic member.

3. An apparatus according to claim 1, wherein said first connecting portion is embedded in an upper portion of the elastic member; and the second connecting portion is formed as a plunger slidably adjustably reciprocated in a central cylindrical hole axially formed in a lower portion of said elastic member; having a longitudinal slot diametrically formed through the elastic member to be communicated with said central cylindrical hole formed in the elastic member.

4. An apparatus according to claim 1, wherein said first connecting portion includes a first cavity axially formed in an upper portion of the first connecting portion adapted for sheathing the first resected fibular section in said first cavity; and said second connecting portion including a second cavity axially formed in a lower portion of the second connecting portion, adapted for sheathing the second resected fibular section in said second cavity.

5. An apparatus according to claim 1, wherein said elastic member, said first connecting portion and said second connecting portion are integrally formed to be a tubular member; said tubular member selected from a solid tubular member and a hollow tubular member.

6. An apparatus according to claim 1, wherein said elastic member is formed as a hollow tubular member, having a through hole radially formed through said tubular member for filling materials into said tubular member for preventing or inhibiting bone fusion or regrowth of the resected fibular sections as held in said tubular member.

7. An apparatus according to claim 1, wherein said elastic member is respectively sheathed in said first and said connecting portions, each said connecting portion formed as a hollow member.

8. An apparatus according to claim 1, wherein said first connecting portion is integrally formed with the elastic portion; and said second connecting portion is slidably sheathed around a lower portion of said elastic member.

9. An apparatus according to claim 1, wherein said elastic member is a hollow tubular member, having an elastomeric column inserted into the hollow tubular member; or filled with silicon rubber or silicon gel into said hollow tubular member.

10. An apparatus according to claim 5, wherein said first connecting portion and said second connecting portion are respectively circumferentially formed thereon with a plurality of extension rings.

11. An apparatus according to claim 1, wherein each said connecting portion is formed with a fixing hole therein, adapted for fixing each said connecting portion to a resected fibular section by a screw.

12. An apparatus according to claim 2, wherein said elastic member includes a plurality of helical spring coils or compressible grooves formed therein; adapted to be compressibly positioned in between the two resected fibular sections, and then expandably fastened to the two said fibular sections.

13. An apparatus according to claim 1, wherein each said connecting portion includes a peg or a projection tapered or protruded outwardly, adapted to be fastened into a medullary cavity in each said resected fibular section.
